(19) United States
(12) Patent Application Publication
(10) Pub. No.: US 2013/0338688 A1
(43) Pub. Date: Dec. 19, 2013

(54) SENSORY VASCULAR CLIP

(71) Applicants: Tausif ur Rehman, Lawrence, KS (US); Atiq ur Rehman, Lawrence, KS (US)

(72) Inventors: Tausif ur Rehman, Lawrence, KS (US); Atiq ur Rehman, Lawrence, KS (US)

(21) Appl. No.: 13/525,447

(22) Filed: Jun. 18, 2012

(51) Int. Cl. A61B 17/122

(52) U.S. Cl. 606/158

(57) ABSTRACT

Method and system is disclosed for using a sensory vascular clip. The sensory vascular clip is configured to open and close and includes first and second clamping portions, each having a pinching surface, a coil spring for pressing said pinching surfaces together when the clip is in a closed position, the coil spring including at least one coil loop, a first and second branch resiliently connected to the coil spring, wherein the first and the second branch cross over each other at a crossing point, a transmitter, and a sensor configured to detect signals from the transmitter.

### TECHNICAL FIELD

This disclosure relates generally to the surgical arts, and more particularly to surgical vascular clips.

### BACKGROUND

The statements in this section merely provide background information related to the present disclosure and may not constitute prior art.

Cerebral aneurysm clips are surgical instruments used for clamping a base part or neck of a cerebral aneurysm to isolate the latter from a cerebral artery. For this purpose, the aneurysm clip must be able to maintain the clamping pressure with high reliability as long as desired without causing injury to the wall of the blood vessel to which it is attached. Such injury could be caused by shearing action resulting from improper alignment of the jaws of the clip, or by improper magnitude of pressure, or by introduction of foreign material trapped in the cracks and crevices of a poorly constructed clip, or by misalignment of a secured clip on a neck of an aneurysm. By way of example, one typical, known surgical aneurysm clip is disclosed in U.S. Pat. No. 4,192,315. The clip includes two jaws and a loop spring mechanism for pressing the jaws together. The spring includes loops that are joined resiliently to each other via a spring loop system.

During surgery, clips allow for the surgeon to clip to the neck of the aneurysm, however, positioning of the clip on the aneurysm neck may be unknowingly off. The surgeon may move the clip off the neck of the aneurysm, leaving the aneurysm viable with blood flow continuing in to the dome of the aneurysm. Additionally, the surgeon may potentially cross the neck of the aneurysm and keep going beyond the neck until he has clipped on other vital blood vessels which are on the other end of the neck of the aneurysm. In such scenarios harm can be caused to the patient. These disadvantages in which the clip has either not gone completely beyond the neck or has gone too far beyond the neck of the aneurysm are dangerous errors that the users of these clips ends up performing, potentially leading to irreversible complications in the brain.

Therefore, there exists a need to provide feedback to the surgeon, enabling communication of positioning indicators of the clip with respect the aneurysm during surgery.

### SUMMARY

It should be apparent therefore that a need still exists in the art of vascular clip that overcomes the aforementioned disadvantages of known prior art clips. Accordingly it is a primary objective of this invention to provide a vascular clip for surgical purposes which can be constructed with dimensions small enough so that it can be used for neurosurgical purposes and can provide a feedback mechanism to the surgeon to allow him/her to place the clip just beyond the end of the aneurysm neck wall so that the aneurysm is occluded and no further vascular or neurological structures are impinged using the clip.

Another object of this invention is to ensure that the vascular clip is connected to a clip application system which can provide the surgeon with good visualization as well as a feedback mechanism which could include visual or an audio or a tactile feedback mechanism to the surgeon to make sure he has gone the correct distance across the neck of the aneurysm.

Therefore, a method and system is disclosed for using a sensory vascular clip. The sensory vascular clip is configured to open and close and includes first and second clamping portions, each having a pinching surface, a coil spring for pressing said pinching surfaces together when the clip is in a closed position, the coil spring including at least one coil loop, a first and second branch resiliently connected to the coil spring, wherein the first and the second branch cross over each other at a crossing point, a transmitter, and a sensor configured to detect signals from the transmitter.

This summary is provided merely to introduce certain concepts and not to identify key or essential features of the claimed subject matter.

### BRIEF DESCRIPTION OF THE DRAWINGS

One or more embodiments will now be described, by way of example, with reference to the accompanying drawings, in which:

FIG. 1 shows a surgical clipping system including a sensory vascular clip, in accordance with the present disclosure;

FIG. 2 shows a perspective view of the sensory vascular clip, in accordance with the present disclosure;

FIG. 3 shows a side view of the sensory vascular clip of FIG. 1, in accordance with the present disclosure;

FIG. 4 shows an enlarged perspective view of a clamping portion of the sensory vascular clip, in accordance with the present disclosure; and

FIG. 5 shows an enlarged side view of the clamping portions of the sensory vascular clip, in accordance with the present disclosure.

### DETAILED DESCRIPTION

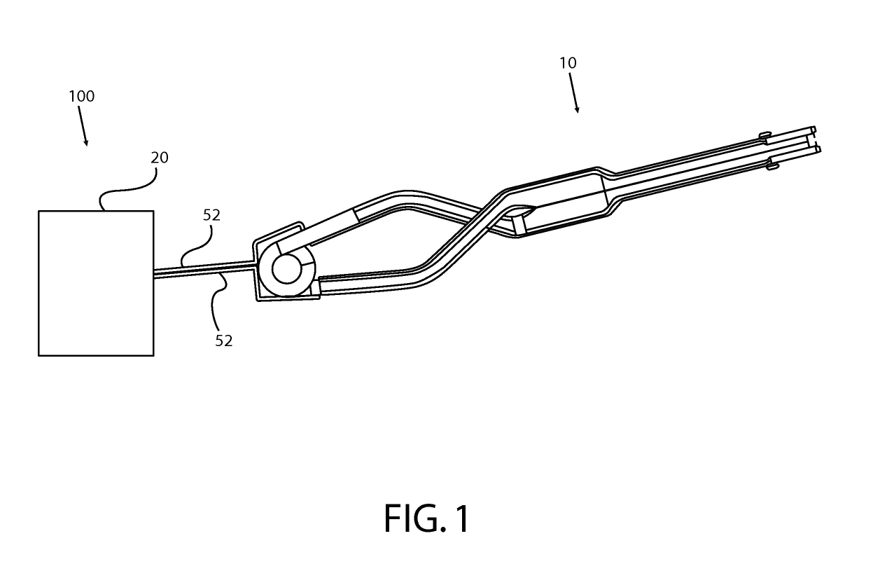

Referring now to the drawings, wherein the depictions are for the purpose of illustrating certain exemplary embodiments only and not for the purpose of limiting the same, FIG. 1 schematically shows a surgical clipping system 100 including a sensory vascular clip 10 and presentation module 20. The system 100 is configured for providing positional feedback of sensory vascular clip 10 with respect to an aneurysm. The clip 10 is communicatively connected to the presentation module 20 via electrical wires or communication cables 52. The presentation module 20 may be one or more types of feedback and/or display system configured to communicate feedback from the sensory vascular clip 10 to a user. In one embodiment, the presentation module 20 is a computer system configured to display images captured via the clip 10 as described herein below. The presentation module 20 may include a microscope view finder. The presentation module 20 may be configured to provide visual, tactile, or audio feedback to a user as described herein below.

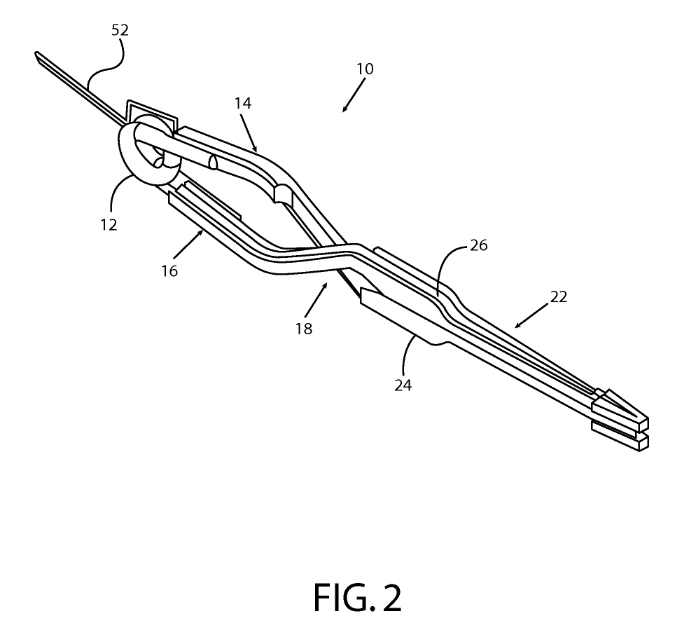

FIG. 2 shows a perspective view of the sensory vascular clip 10. The clip 10 may be formed of a resilient material which is bent so as to form a double coil 12 and two branches 14, 16 that are mutually resiliently pivotal. In one embodiment, the clip 10 may be fabricated from titanium metal or a titanium alloy. The titanium alloy is a suitable material since it is inert, compatible with human body fluid and tissue and non-magnetic. The clip 10 may be formed by first stamping an elongated resilient member from a solid rod of titanium or titanium alloy having, for example, a diameter of about 35 mils and a length of about 2 inches. It should be noted that these particular parameters for the clip 10 here described are exemplary, and the invention is in no way limited to fabrication of clips in this particular size range. The branches 14 and 16 are bent towards one another at an angle in the plane of the coil 12 so that the two branches 14 and 16 cross at a cross-over region 18. After the cross-over region 18, the branches 14 and 16 are bent towards one another and meet each other frontally in a clamping region 22. Each of the branches 14 and 16 end in two clamping portions 24 and 26 which abut one another and project preferably parallel with a plane of the coil 12 and the branches 14 and 16. The clamping portions 24 and 26 are pressed resiliently together due to the spring effect of the coil 12 and they can only be separated from one another by a resilient deformation of the vascular clip 10.

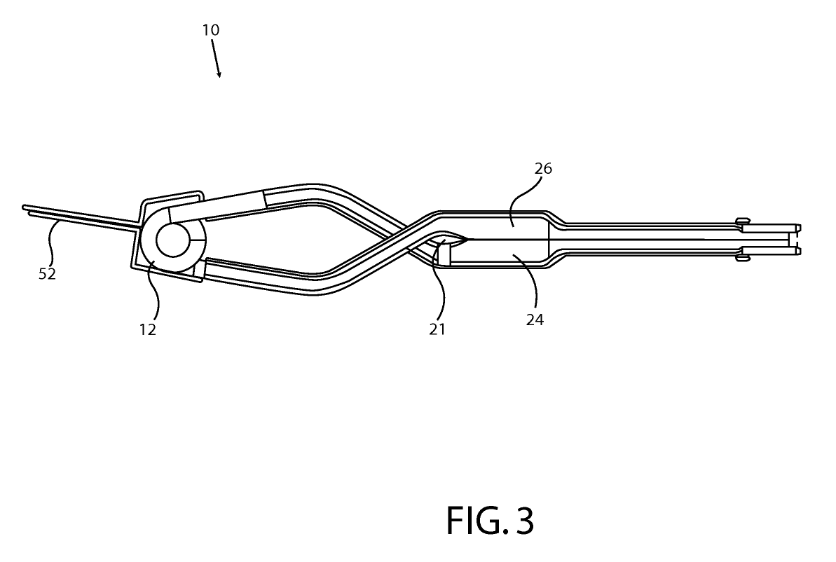

FIG. 3 shows a side view of the sensory vascular clip of FIG. 1. As FIG. 3 shows, the sides of the clamping portions 24 and 26 which face each other are formed as pinching surfaces 25. Alternately fitting recesses may be provided on the ends of the pinching surfaces 25. As seen in FIG. 1, when the pinching surfaces 25 press against one another, the parts of the clamping portions 24 and 26 are spaced from each other so that, between the cross-over region 18 and the pinching surfaces 25, an opening 21 is formed.

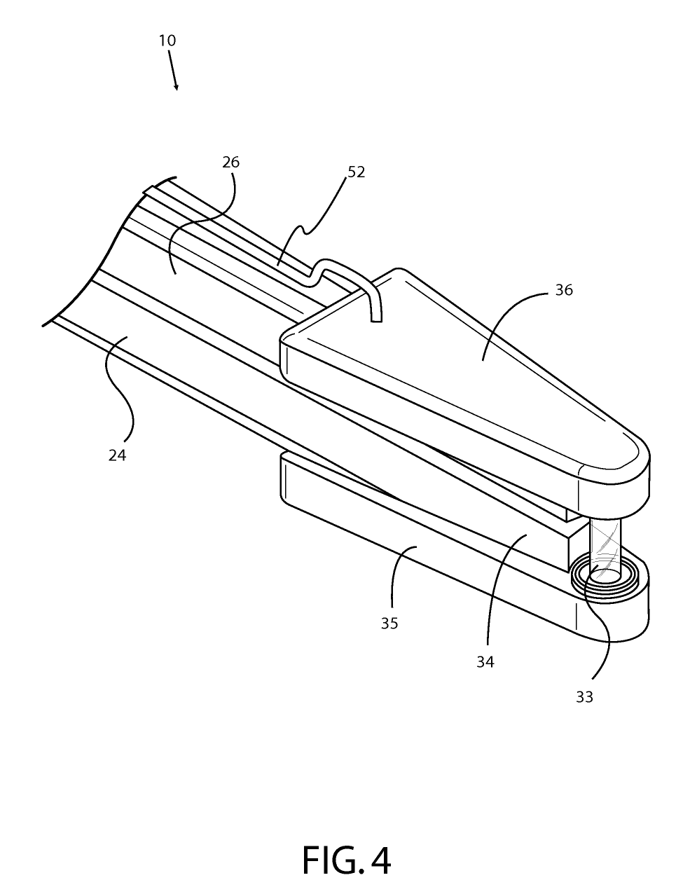

FIG. 4 shows an enlarged perspective view of the clamping portions 24 and 26 of the sensory vascular clip 10. As FIG. 4 shows, the clamping portions 24 and 26 include end portions 34 and 36. The end portions 34 and 36 are respectively attached to the clamping portions 24 and 26. In one embodiment, the end portions are respectively integrally connected to the clamping portions 24 and 26. The end portions 34 and 36 overlap ends 33 and 35 of the clamping portions 24 and 26.

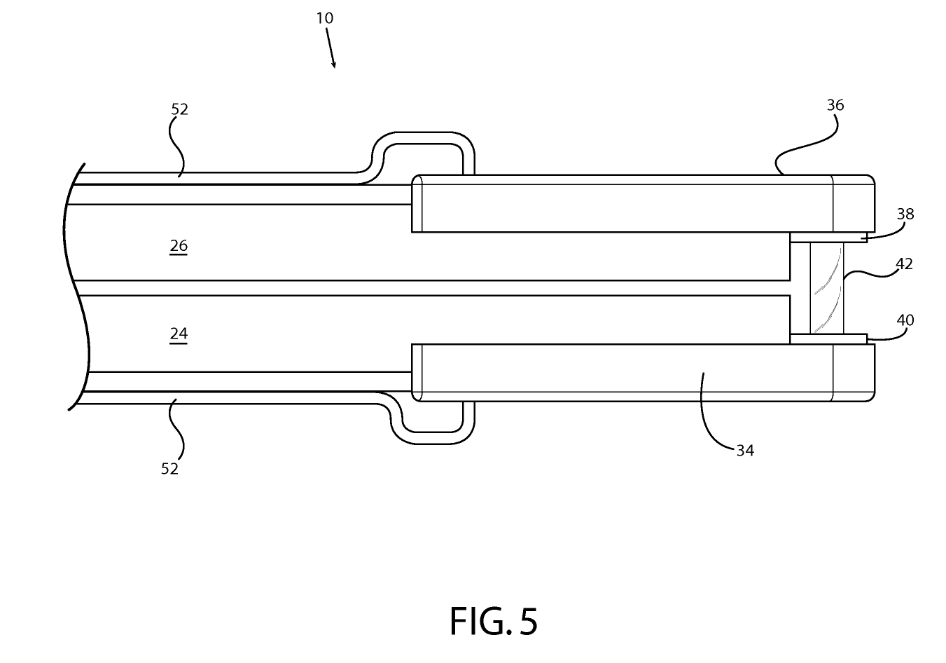

FIG. 5 shows an enlarged side view of the clamping portions 24 and 26 of the sensory vascular clip 10. As FIG. 5 shows, a transmitter 38 is affixed to the end portion 36 and a sensor 40 is attached to the end portion 34. The transmitter 38 may be one of multiple known types of emitters including electromagnetic, ultrasonic, or light-based such as a laser light device or infrared-based device. In a preferred embodiment of the disclosure, the clamping portions 24 and 26 of the clip 10 in a position lying against one another are able to provide a light emitted from the tip of one of the clamping portions 24 and 26 which is detected by the tip of the other clamping portions 24 and 26 once is has crossed the aneurysm wall boundaries. This then creates a feedback to a surgeon so that the clip 10 may be deployed once the aneurysm wall has been passed when operating. In operation, the transmitter 38 emits an emission 42 toward the sensor 40. Upon detection of the emission 42, the sensor completes a feedback loop to the presentation device described herein above. Upon interruption of the emission 42, the feedback loop is broken and a feedback response is generated for presentation to the user.

FIG. 6 shows an enlarged side view of an additional embodiment of the clamping portions 24 and 26 of the sensory vascular clip 10 having an imaging device 60 disposed thereon. As FIG. 6 shows, the imaging device 60 may be affixed to the end portion 36. In one embodiment, a lighting device 62 is utilized to illuminate a proximate area in a field-of-view of the imaging device 60. The imaging device 60 may be configured to capture visible and invisible light for image processing including known filtering techniques for presentation to the user via the presentation module 20.

The disclosure has described certain preferred embodiments and modifications thereto. Further modifications and alterations may occur to others upon reading and understanding the specification. Therefore, it is intended that the disclosure not be limited to the particular embodiment(s) disclosed as the best mode contemplated for carrying out this disclosure, but that the disclosure will include all embodiments falling within the scope of the appended claims.

1. A vascular clip having open and closed positions comprising:
a first and a second clamping portion, each having a pinching surface;
a coil spring for pressing said pinching surfaces together when the clip is in a closed position, the coil spring including at least one coil loop;
a first and a second branch resiliently connected to the coil spring, wherein the first and the second branch cross over each other at a crossing point;
a transmitter; and
a sensor configured to detect signals from the transmitter.

2. The vascular clip of claim 1, further comprising:
a first lip attached to an end of the first clamping portion; and
a second lip attached to an end of the second clamping portion.

3. The vascular clip of claim 2, wherein the transmitter is affixed to a surface of the first lip and the sensor is affixed to a surface of the second lip.

4. The vascular clip of claim 1, further comprising:
a first wire connected to the transmitter; and
a second wire communicatively connected to the sensor.

5. The vascular clip of claim 1, wherein the transmitter is a light emitting device.

6. The vascular clip of claim 1, wherein the transmitter is an electromagnetic radiating device, and wherein the sensor is an antenna configured to detect emissions from the electromagnetic radiating device.

7. The vascular clip of claim 1, wherein the sensor is configured to communicate a discrete signal based upon transmissions from the transmitter.

8. A surgical clipping system, comprising:
a sensory vascular clip configured to detect whether clamping portions of the sensory vascular clip have passed around an aneurysm; and
a presentation device configured to communicate feedback from the sensory vascular clip to a user.

9. The system of claim 8, wherein the sensory vascular clip comprises:
a first and a second clamping portion, each having a pinching surface;
a coil spring for pressing said pinching surfaces together when the clip is in a closed position, the coil spring including at least one coil loop;
a first and a second branch and resiliently connected to the coil spring, wherein the first and the second branch cross over each other at a crossing point;
a transmitter; and
a sensor configured to detect signals from the transmitter.

10. The system of claim 8, wherein the presentation device is a visual display.

11. The system of claim 8, wherein the presentation device is a tactile feedback device.

12. The system of claim 8, wherein the presentation device is configured to generate an audio response.

13. The system of claim 8, wherein the sensory vascular clip comprises an imaging device.

14. The system of claim 13, wherein the imaging device is communicatively connected to the presentation device.

15. The system of claim 8, wherein the sensory vascular clip comprises:
a first and a second clamping portion, each having a pinching surface;
a coil spring for pressing said pinching surfaces together when the clip is in a closed position, the coil spring including at least one coil loop;
a first and a second branch and resiliently connected to the coil spring, wherein the first and the second branch cross over each other at a crossing point;
a first end portion; and
an imaging device affixed to the first end portion.

16. A method for clipping an aneurysm, the method comprising:
identifying an aneurysm;
opening a sensory vascular clip;
positioning the sensory vascular clip around a base portion of the identified aneurysm;
receiving positioning feedback from the sensory vascular clip; and
closing the sensory vascular clip on the base portion of the identified aneurysm based upon the received positioning feedback.

17. The method of claim 16, wherein the positioning feedback comprises visual imaging information.

18. The method of claim 16, wherein the positioning feedback comprises a tactile response.

19. The method of claim 16, wherein the positioning feedback comprises an audio response.

20. The method of claim 16, further comprising:
transmitting an interruptible signal; and
detecting the base portion of the identified aneurysm based upon an interruption in the signal.
